(19) United States
(12) Patent Application Publication
(10) Pub. No.: US 2026/0240641 A1
(43) Pub. Date: Aug. 20, 2026

(54) HYBRID RETRIEVABLE STENTGRAFT AND SHUNT FOR PERFUSION PRESERVING HEMORRHAGE CONTROL

(71) Applicant: Ohio State Innovation Foundation, Columbus, OH (US)

(72) Inventor: Bryan TILLMAN, Delaware, OH (US)

(21) Appl. No.: 19/489,628

(22) PCT Filed: Jun. 5, 2024
(86) PCT No.: PCT/US2024/032504

§ 371 Date: Dec. 3, 2025

Related U.S. Application Data
(60) Provisional application No. 63/506,192, filed on Jun. 5, 2023.

(51) Int. Cl. A61F 2/07; A61F 2/97; A61L 29/04; A61M 25/06; A61M 25/09

(52) U.S. Cl. CPC A61F 2/07; A61F 2/97; A61L 29/041; A61M 25/065; A61M 25/09

(57) ABSTRACT

A Hybrid Rescue Shunt (HRS) that includes a retrievable stentgraft on one end to provide hemorrhage control while simultaneously providing blood inflow to a distal multi-cannula that includes a distal shunt that is connected to the stentgraft. The HRS further includes a peel-away sheath, a nosecone and integrated guidewire to aid in the deployment of the HRS within vessel. The HRS may be inserted separate from the injury or surgical site, allowing placement during compression of the actual injury or when the surgical site is not well visualized.

### CROSS-REFERENCE TO RELATED APPLICATIONS

This application claims priority to U.S. Provisional Patent Application No. 63/506,192, filed Jun. 5, 2023, entitled “HYBRID RETRIEVABLE STENTGRAFT AND SHUNT FOR PERFUSION PRESERVING HEMORRHAGE CONTROL,” the disclosure of which is expressly incorporated herein by reference.

### BACKGROUND

Surgical techniques to repair vascular damage typically rely on use of clamps (or occasionally occlusive intravascular balloons) to achieve hemorrhage control during repairs, which threatens perfusion of downstream of the repair. Also, because the vascular occlusion causes backpressure above the repair this causes stress on the heart. For many tissues, beyond 20 minutes of absent blood flow serious tissue damage (ischemia) can occur including Renal failure, Bowel ischemia, Liver failure, Paralysis, and Release of toxic electrolytes and muscle proteins from dying tissue. Also, the increased backpressure above the clamp can separately cause myocardial ischemia (heart attack) and acute lung injury.

### SUMMARY

A Hybrid Rescue Shunt (HRS) that includes a retrievable stentgraft on one end to provide hemorrhage control while simultaneously providing blood inflow to a distal multi-cannula that includes a distal shunt that is connected to the stentgraft. The HRS further includes a peel-away sheath, a nosecone and integrated guidewire to aid in the deployment of the HRS within vessel. The HRS may be inserted separate from the injury, allowing placement during compression of the actual injury in traumatic injuries or in elective aortic surgery when clamp placement may be otherwise difficult as related to scar tissue, tumor or vessel inappropriate for clamp placement.

The present disclosure describes various implementations that achieve “perfusion preserving hemorrhage control,” thereby avoiding both distal ischemia and proximal back pressure. This in turn improves surgical outcomes and patient safety during complex elective and emergent vascular repairs. In addition, as aspects of the present disclosure replaces the vascular clamp, they prevent clamp related iatrogenic injuries which includes injury of adjacent structures and difficulties and injuries from clamping calcified vessels. Yet further, as aspects of the present disclosure do not require exposure of adjacent vessel for a clamp placement, they prevent complicated additional vessel exposure within the hostile abdomen (prior surgery or scar tissue, or bulky tumor). Still further, despite different locations in the body, vascular injuries of the torso and junctional regions are described as sharing a similar solution.

Thus, in accordance with the above, disclosed herein is a device that includes a proximal stentgraft, a nosecone that is integrated with the stentgraft, a guidewire that extends from a distal end of the nosecone, a distal shunt attached to the stentgraft, and a sheath that surrounds the stentgraft and distal shunt.

In accordance with other aspects, disclosed herein a device that includes a proximal flexible stentgraft having a distal tapered portion, the flexible stentgraft being encapsulated in a polymer; and a distal shunt that includes a manifold and a plurality of cannula connected to the manifold. The flexible stentgraft provides hemorrhage control while simultaneously providing blood inflow to the distal shunt and one or a plurality of cannula.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIGS. 1A-1E illustrate a Hybrid Rescue Shunt (HRS) in accordance with aspects of the disclosure;

FIGS. 2A-2D illustrate an example process by which a stentgraft may be constructed in accordance with aspects of the disclosure;

FIGS. 3A-3B illustrate a stentgraft within a sleeve as part of an example delivery system in accordance with aspects of the disclosure;

FIGS. 4A-4D illustrate an example implementation of a manifold and cannula of the HRS in accordance with aspects of the disclosure;

FIGS. 5A-5C illustrate aspects of stent diameter and cell size;

FIG. 6 illustrates a seal zone and suturing zone provided by the HRS in accordance with aspects of the disclosure;

FIGS. 7A-7C illustrates a split introducer (SI) needle in accordance with aspects of the disclosure;

FIGS. 8A-8D illustrate an example application of the HRS during open aortic surgery in accordance with aspects of the disclosure;

FIG. 9 illustrates example organs and target areas that may be perfused by each cannula in accordance with aspects of the disclosure;

FIGS. 10A-10C illustrate additional details of an example application of the HRS during open aortic surgery in accordance with aspects of the disclosure;

FIGS. 11A-11D and 12A-12D illustrate an example application of the HRS during a staged damage control procedure in accordance with aspects of the disclosure; and

FIGS. 13A-13C illustrate an example application of a modified version of the HRS for junctional hemorrhage in accordance with aspects of the disclosure.

### DETAILED DESCRIPTION

A stent is a tubular cage of metal used to strut open diseased vessels. A “stentgraft” is distinguished by a polymer covering, allowing use in redirecting blood flow such as in repair of degenerated (aneurysmal) or injured vessels where a fluidic seal against the vessel wall is required to exclude blood flow. Retrievable stentgrafts are distinct from permanent stentgrafts in that retrievable stentgrafts can be removed after use. However, a notable limitation of the retrievable stentgraft alone as an adjunct to open surgery is near vascular branches such as the visceral aorta or pelvis in the torso or alternately, in junctional regions where the vessels may branch. Further, a shortcoming of current shunts is the need to place a tubular shunt into a vessel injury while it is bleeding and without proximal hemorrhage control. The present disclosure overcomes these limitations and provides other advantages.

### Hybrid Rescue Shunt (HRS)

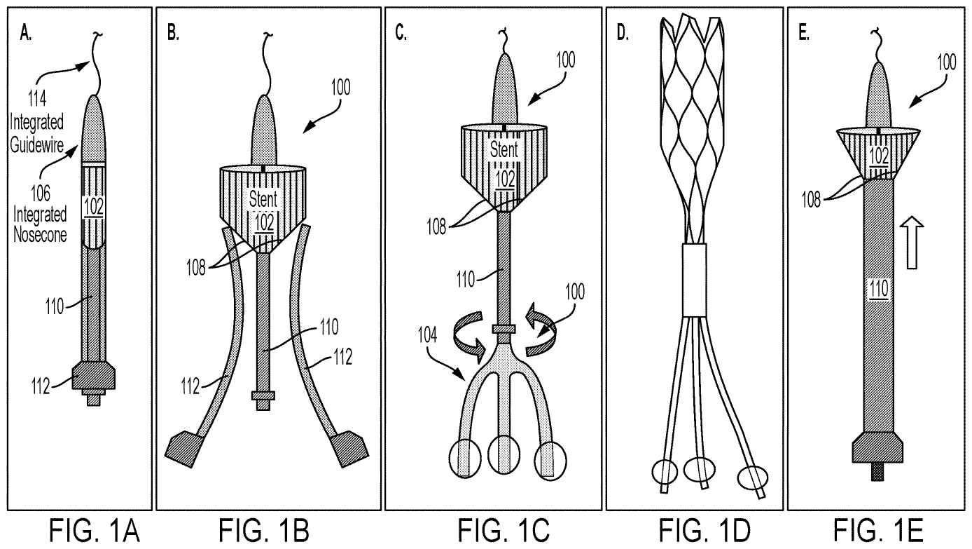

In accordance with an aspect of the disclosure, FIGS. 1A-1E illustrate a Hybrid Rescue Shunt (HRS) 100 that is provided with a flexible retrievable stentgraft 102 on one end to provide hemorrhage control while simultaneously providing blood inflow to a distal shunt 110 that is connected to the stentgraft 102 that includes multiple cannulae 104. The distal shunt 110 and cannula 104 are provided for distal perfusion, as described below. The HRS 100 further includes a nosecone 106 and an integrated guidewire 114 to facilitate passage through a vessel wall or skin. A peel away sheath 112 is provided to deploy the HRS 100. The use and deployment of the HRS 100 is described below in several example scenarios.

An advantage of the HRS 100 over a standard tubular shunt is that the HRS 100 can be inserted remote from the injury, allowing placement during compression of the actual injury. To achieve this, the integrated nosecone 106 is attached to the stentgraft 102 upon vessel access to provide a nearly single-step delivery of the HRS 100 for hemorrhage control. The HRS 100 may be delivered as a single component or two components that are connected to each other depending on the clinical use.

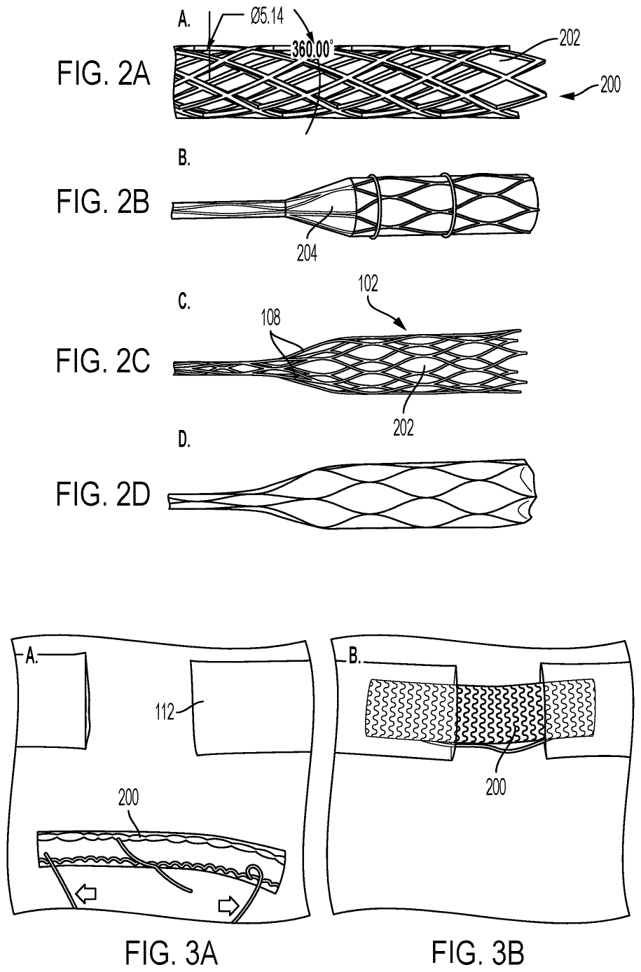

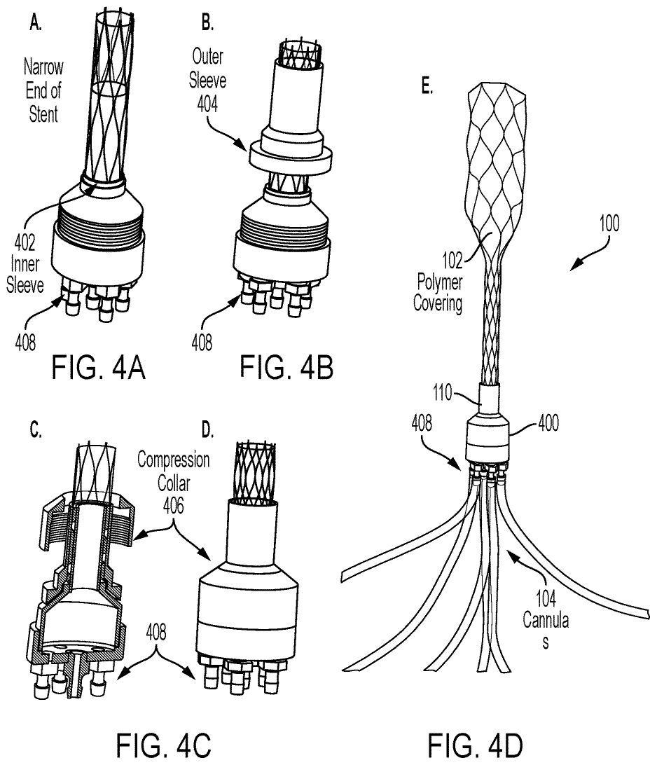

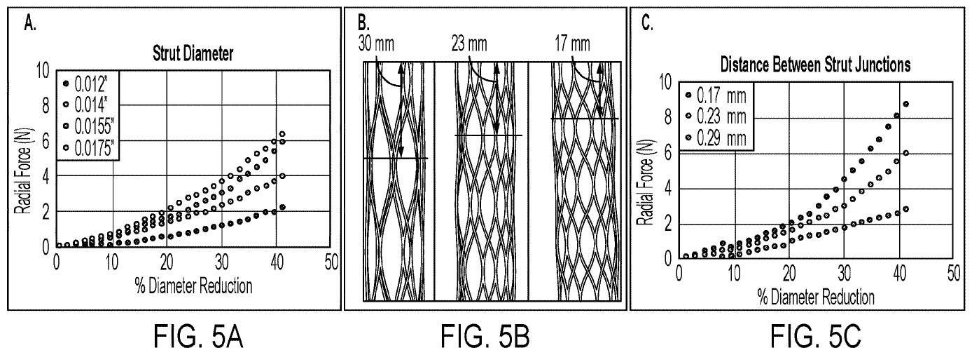

With reference to FIGS. 2A-2D, there is illustrated an example process by which the stentgraft 102 may be constructed. Designs may be developed within 3D computer aided design (CAD) software (e.g., Solidworks, Waltham, MA) followed by laser cutting (Confluent Medical, Scottsdale, AZ) to make the stentgraft 102. As shown in FIG. 2A, a scaffold 200 defining a plurality of cells 202 may be thermally shapeset using a mandrel 204 having a tapered cylinder shape (FIG. 2B) to define the general shape of the stentgraft 102 such that is has a tapered portion 108 (FIG. 2C). The stentgraft 102 is then encapsulated in the polymer (e.g., polytetrafluoroethylene) and permanently attached to a luer lock connector (FIG. 2D). Although not shown, during assembly, the nosecone 106 may be integrated with the stentgraft 102. The distal end may be constructed to include 2-4 balloon tipped irrigation cannulas (example Pruitt, LeMaitre Vascular. Burlington, MA), using a ¾-way connector 408 (see, FIGS. 4A-4D) affixed to a matching luer connection. An example delivery system is shown in FIGS. 3A-3B, wherein the peel away sheath 112 may be constructed of polyethylene or polytetrafluoroethylene.

Alternately for elective surgery the stent and shunt may be fabricated into a single piece. FIGS. 4A-4D illustrate details of the distal shunt 110 in greater detail and provide an example implementation of a manifold 400 and cannula 104. Generally, the manifold 400 connects stentgraft 102 to the cannulas 104 to provide blood flow from a vessel to the cannulas 104. An inner sleeve 402 is disposed inside the stentgraft 102 and is paired with an outer sleeve 404. A compression collar 406 compressively fixes the outer sleeve 404 to the inner sleeve 402, as shown in FIG. 4C.

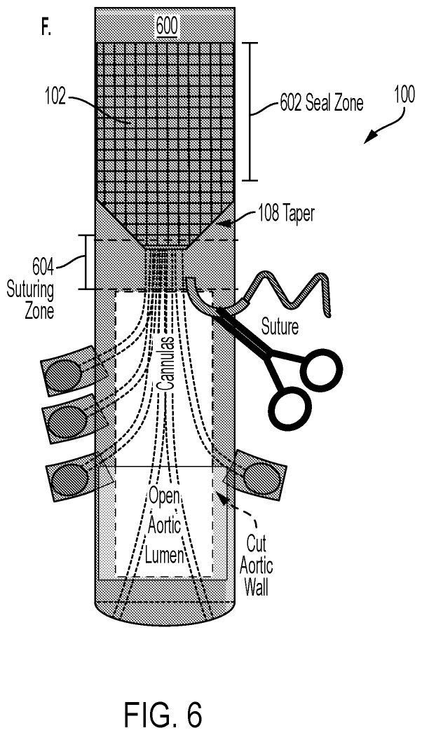

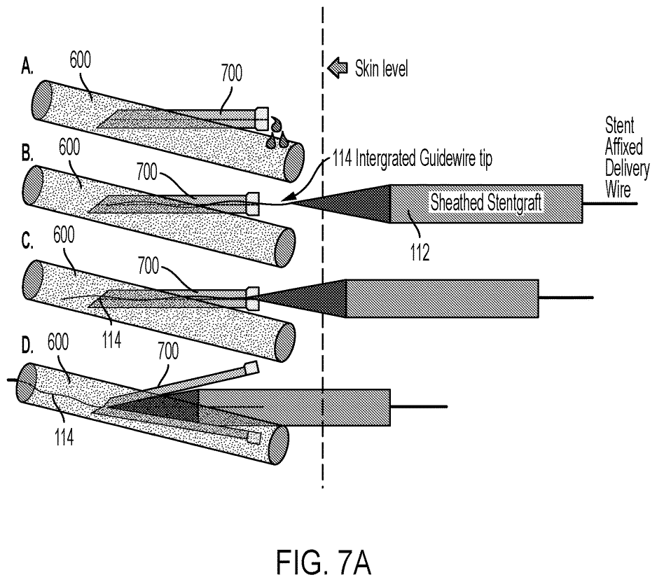

As shown in FIGS. 5A-5C, a stent strut diameter and stent cell size are related to improvement of radial force of the stentgraft 102 that creates a fluidic seal against the vessel wall. In accordance with the example processes of FIGS. 2A-2D, FIG. 6 shows that the stentgraft 102 and tapered portion 108 creates a seal zone 602 and suturing zone 604 within a vessel 600 into which the stentgraft 102 is inserted.

### Split Introducer (SI) Needle

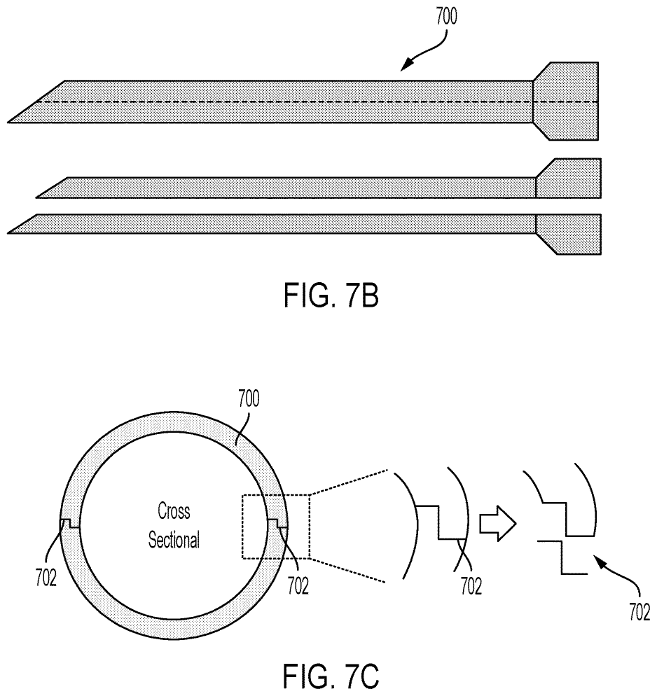

With reference to FIGS. 7A-7C, there is illustrated a split introducer (SI) needle 700 in accordance with another aspect of the disclosure. The SI needle 700 simplifies endovascular delivery in an emergency. Traditional vascular access requires a cumbersome series of steps the Seldinger technique including wire access, placement of a wire, placement of a larger delivery sheath and insertion of the sheathed stent device. The solution of the present disclosure reduces steps and simplifies rapid delivery by using the novel split needle 700 as shown in FIG. 7A. In this way, the needle 700 is used for vessel access as normal (see, FIG. 7A, A), but the guidewire 114 of the sheathed stent is placed through the needle 700 (see, FIG. 7A, B, C). Advancing the device 114 and 112 splits the needle 700 in half while the device is advanced into a vessel 600 (see, FIG. 7A, D). This avoids steps of a separate guidewire and second sheath. Additionally, the absence of a second sheath reduces the overall delivery size of the device. FIG. 7B further details that the SI needle 700 generally splits in half along a longitudinal axis. FIG. 7C illustrates a cross-sectional view of the end of the SI needle 700 of FIG. 7B., and illustrates an example joint 702 that is formed between halves of the SI needle 700.

### Example Use Cases of the HRS and/or SI Needle

### Application of the Implementations of the Disclosure for Aortic Injury

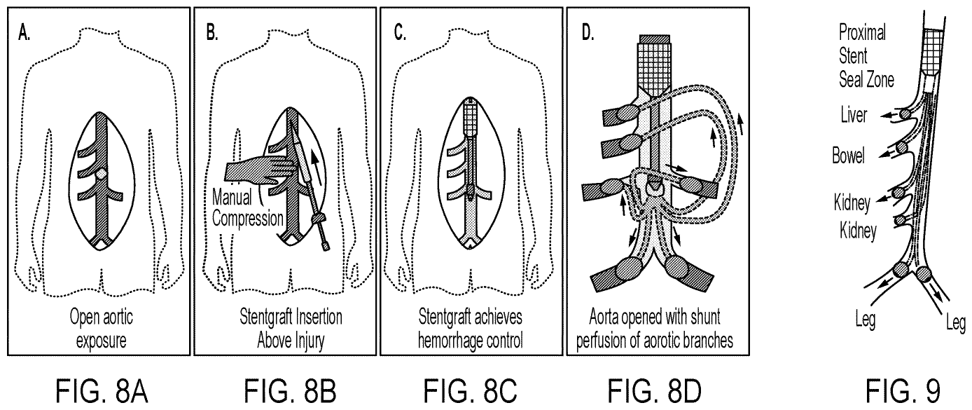

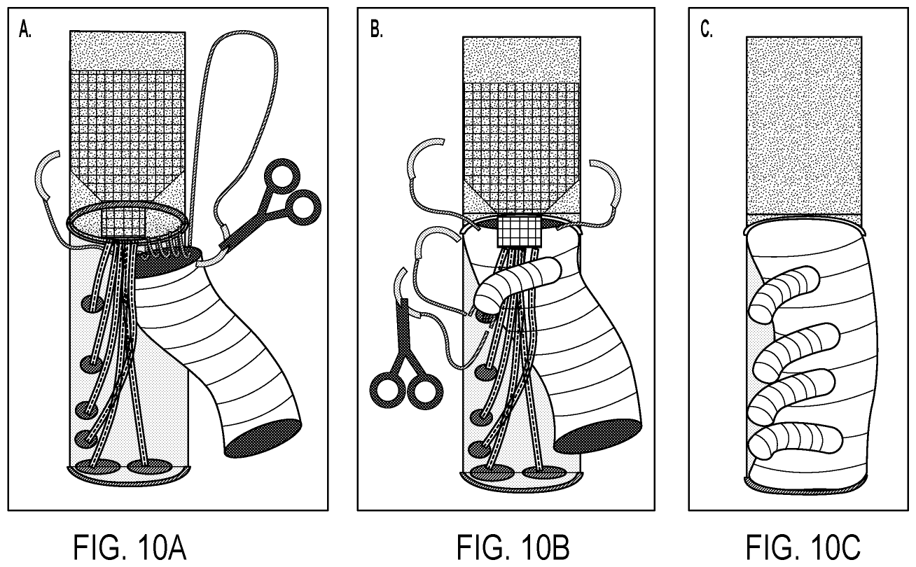

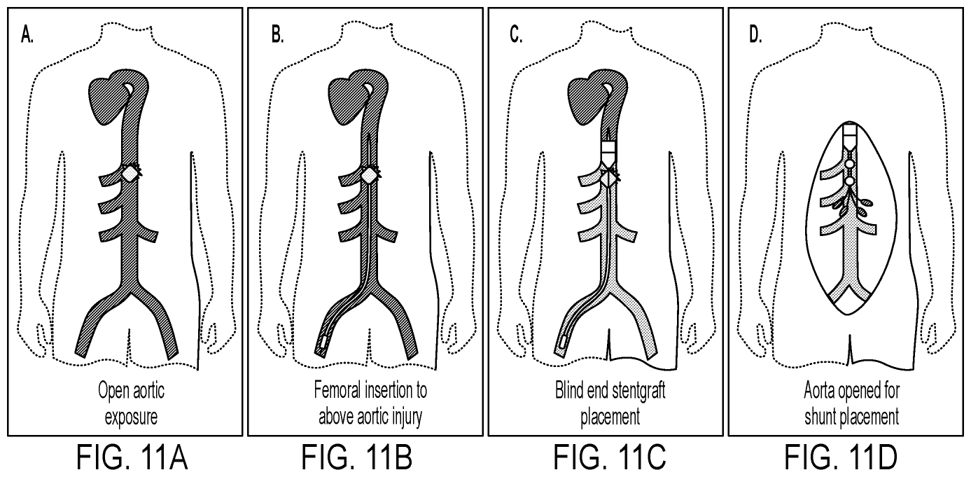

With reference to FIGS. 8A-8D, 9 and 10A-10C, there is illustrated an example application of the HRS 100 during open aortic surgery. For aortic injuries, there may be two non-limiting delivery modes for the HRS 100. If the abdomen is opened surgically for damage control of other injuries such as gastrointestinal injuries, direct compression would be held on the injured aorta, and then the HRS would be inserted directly through the aortic wall proximal to the injury, with deployment of a retrievable stentgraft to achieve proximal hemorrhage control. The blind end of the proximal stentgraft component may then be connected to the distal multi-lumen cannulae component and used for distal perfusion (see, FIG. 8D). The reasoning for accessing uninjured aorta above the injury is that placement of a device through an injury is hindered by pulsatile flow obscuring visualization whereas applying proximal compression is often more challenging given the anatomy in this area. Nevertheless, the device can also be inserted through the injury if that is more favorable. FIG. 9 illustrates example organs and target areas that may be perfused by the cannula. FIGS. 10A-10C illustrate that the aorta is opened and perfusion cannulas are placed in branches and a draft is sewn starting with a posterior side of the shunt. When proximal anastomosis is substantially complete, branch grafts are then sewn on moving flexible shunt as needed. As shown in FIG. 10C, the shunt is removed and suture lines completed after flushing.

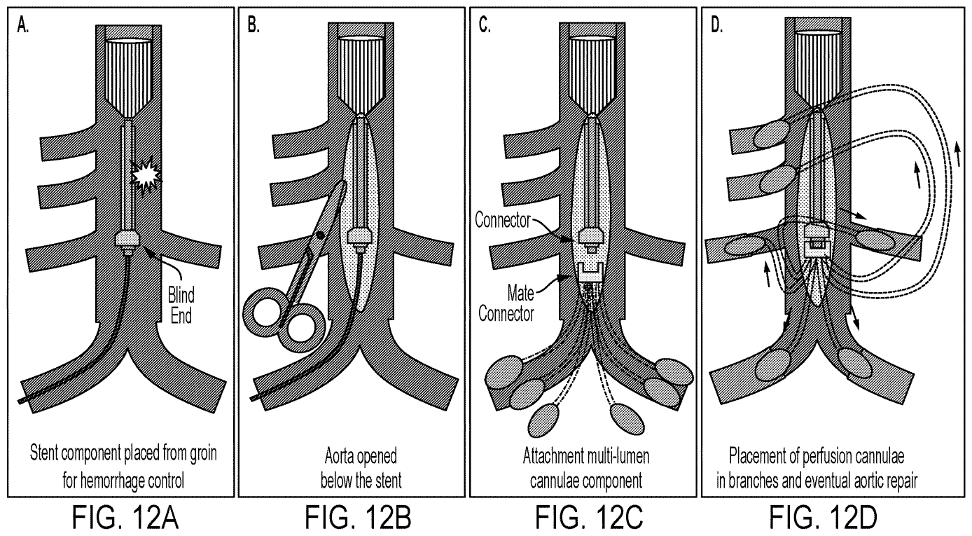

Alternately, if the environment does not favor immediate open exploration, the same stentgraft component device could be used in a “Staged Damage Control” approach for the even more austere environment, with placement percutaneously from the groin and deployment above the injury. With reference to FIGS. 11A-11D and 12A-12D, a blind end of the stentgraft provides immediate hemorrhage control similar to a balloon occlusion to address the most immediate threat to life. The stiff delivery wire still attached to the blind end of the stentgraft and emerging from the groin access provides fixation of the device against intense aortic pulsation that otherwise may dislodge the stentgraft. Later, when the injured patient has been moved to the next Echelon of care, the patient would proceed to abdominal exploration, opening of the aorta to expose the blind stentgraft end and attachment of the shunt end for distal reperfusion. This distal shunt could be placed in individual visceral arteries, the distal aorta, or iliac arteries thus providing maximal flexibility for a number of injury patterns. If less cannula are needed the extra cannula can simply be clamped.

### Application of the Implementations of the Disclosure for Junctional Hemorrhage

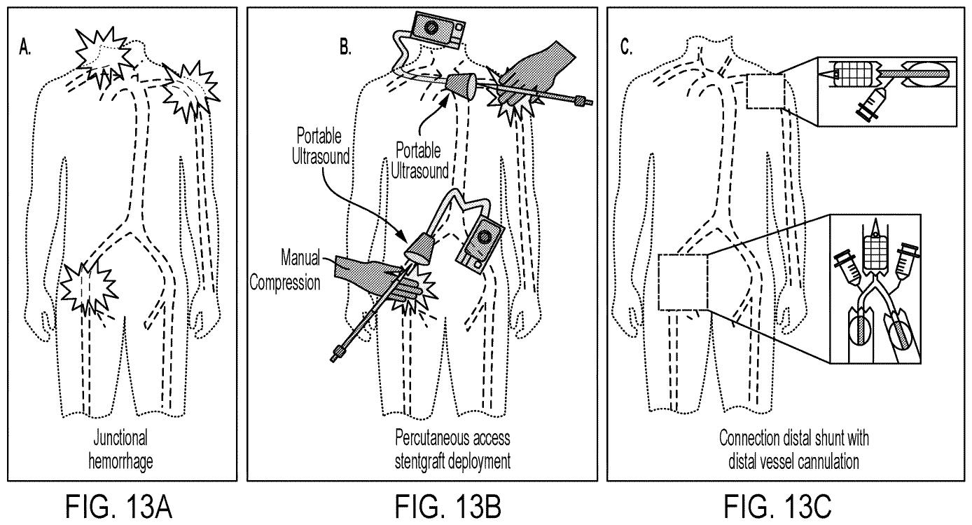

With reference to FIGS. 13A-13C, for junctional hemorrhage, a smaller version of the HRS can provide a similar theme of proximal hemorrhage control and distal multi-cannula shunt. Although the HRS could certainly be placed through an open proximal vessel lumen from the groin or shoulder, junctional hemorrhage by definition poses challenges that the vessel retracts into the less accessible torso. For this reason, a percutaneous approach, intentionally targeting the iliac or subclavian artery provides proximal vessel access for the combined hemorrhage control and shunt inflow. After proximal hemorrhage control is complete the end of the retrievable stentgraft is now attached to a multi-cannula shunt which is placed into one or multiple distal vessels to restore distal perfusion. A key advantage of the HRS is the ability to place the device proximal to the injury and thereby avoid placement of a shunt into an actively hemorrhaging injury without proper control of the proximal vessel. By contrast, it would not be possible to achieve percutaneous access with currently available tubular or balloon tipped shunts.

### Device Removal

In accordance with aspects of the disclosure, the HRS 100 is not intended as a permanent repair, but rather as a tool to simplify open elective aortic repair and a rapid damage control of vascular injuries. Whereas traditional stentgrafts are intended as a permanent implant, the stentgraft of the HRS is retrievable. For example, at the time of definitive repair in a higher Echelon of care, the stentgraft is removed. Specifically, the HRS can be recaptured by sheath advancement to collapse the stentgraft for removal and the access can be closed by a simple suture repair or permanent stentgraft as appropriate.

### Example Critical Care Use Case

In environments, such as battlefields, injuries of the torso and those at the interface of the torso and extremities (junctional hemorrhage) represented 60% of fatalities. Traumatic injuries of the aorta, especially proximally and around branches to abdominal organs require significant expertise and resources that are not available in the austere environment, further complicated by the imminent risk of exsanguination and by delays in transport to Echelon 4 and 5 for definitive surgical repair. Junctional injuries occur at the interface of an extremity (or neck) and the torso, representing 8.5% of all extremity injuries, as reported from the Iraq and Afghanistan Wars. The location of these injuries near the torso pose significant barriers in terms of proximal control given the more complex surgical exposures required. Junctional tourniquets, which are relatively new, apply focal pressure to mitigate hemorrhage and yet risk ischemia/reperfusion in the extremity during prolonged use with loss of a functional outcome. Thus, complications of aortic occlusion for damage control and challenges to inflow of junctional injuries remain threats to life and limb for injured soldiers. In both scenarios, the HRS 100 of the present disclosure would provide a better approach and would improve outcomes.

### Example Benefits of the HRS for Elective Surgery Uses

In the United States alone, there remain over 17,000 elective open aortic repairs performed each year, even in the era of minimally invasive repair. Such surgical procedures include, but are not limited to, aneurysm repairs, vascular invasive tumors, and explant of failed permanent stengrafts. Typically, Aortic Crossclamp (ACC) is used. However, in surgical repairs that require a clamp above the visceral aorta, e.g., branches to the bowel, liver and kidneys, this procedure threatens ischemic injury and organ failure due to the number of vessels that must be reperfused in sequence. Other consequences, such as paralysis and direct clamp injury to nearby structures such as the pancreas and esophagus are possible.

Conventional removeable stentgrafts are also used for aortic repairs. However, the use of removeable stentgrafts faces barriers for elective repairs of branched aorta. Some of the barriers include: 1) The lower aorta is often too diseased to seal a stent. 2) Whereas a stent contains one inflow and one outflow, branches of complex aortic repair require multiple outflows to cannulate each branch. 3) A stentgraft is inflexible to diverse aortic lengths and would obligate significant inventory.

The HRS of the present disclosure provides several advantages for elective, open aortic surgical procedures. In particular, the HRS can provide for a bloodless open surgical field. The HRS improves perioperative vascular physiology and acidosis over clamping. The HRS markedly improves distal visceral flow over ACC. The HRS markedly improves distal visceral flow over ACC. The HRS prevents organ ischemia. Thus, the HRS provides benefits for use in elective, open aortic surgical procedures in addition to those in trauma situations.

Contrasting to the placement of stent component first and later a staged connection to a shunt component, when used for elective aortic repair with no traumatic injury, a single piece HRS device (stent already connected to the multi-cannula shunt) would most likely be used. In that case the stent end would be placed through the aortic wall above the level of the expected repair site with subsequent deployment of the stent end, follow by immediate placement of the cannulae into target branch vessels.

### CONCLUSION

The following is a non-limiting list of procedures that may benefit from the various implementations of the present disclosure: repair of traumatic injuries (aortic and junctional). (junctional are injuries where the torso meets the extremity), repair of complex aneurysms (especially those near major branches in the chest or abdomen), resection of complex cancers that encase or invade a major blood vessel, and repair of infected major vessels.

Further, the HRS 100 may avoid placement of the shunt through the injury itself, thereby avoiding excessive operative exposure, hemorrhage, or difficulty securing the shunt. Rather the stent component forms a vascular seal and hemorrhage control proximally. Also, although stents and X-ray imaging often go hand in hand, in this setting the HRS is inserted by markers on the device and does not require X-ray imaging that is impractical for an austere environment.

Thus, the HRS accomplishes aortic and junctional hemorrhage control, and the proximal stentgraft component serves as proximal inflow for the distal shunt component thereby preventing ischemia/reperfusion complications and improving outcomes. Further damage control capabilities and solutions for control of non-compressible torso hemorrhage, especially for austere environments may be developed.

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A device, comprising:
a proximal stentgraft;
a nosecone that is integrated with the stentgraft;
a guidewire that extends from a distal end of the nosecone;
a distal shunt attached to the stentgraft; and
a sheath that surrounds the stentgraft and distal shunt.

2. The device of claim 1, wherein the stentgraft is flexible.

3. The device of claim 2, wherein the stentgraft further includes a distal tapered portion.

4. The device of claim 3, wherein the distal tapered portion of the stentgraft creates a seal zone within a vessel which the flexible stentgraft is inserted and a suture zone at a narrowed stent segment.

5. The device of claim 1, wherein the stentgraft provides hemorrhage control while simultaneously providing blood inflow to the distal shunt.

6. The device of claim 1, wherein the sheath peels-away during deployment of the device within a vessel.

7. The device of claim 6, wherein the sheath is composed of polyethylene or polytetrafluoroethylene.

8. The device of claim 1, wherein the stentgraft is removable by sheath advancement to collapse the stentgraft.

9. The device of claim 1, wherein the device is adapted to be inserted into a patient's body at a location that is separate from an injury or elective surgical location.

10. The device of claim 1, further comprising a split introducer needle, wherein the guidewire is placed through the split introducer needle, and wherein advancing the device guidewire splits the split introducer needle in half while the device is advanced into a vessel.

11. The device of claim 1, further comprising a one or a plurality of cannula connected to the distal shunt.

12. The device of claim 11, wherein the device is deployed into an aorta of a patient and wherein the plurality of cannula provide blood flow to organs and predetermined target areas in the patient's body.

13. The device of claim 11, wherein the device is deployed for a junctional hemorrhage.

14. The device of claim 11, wherein the distal shunt includes a manifold having an inner sleeve that is enclosed inside the stentgraft and an outer sleeve, wherein a compression collar compressively fixes the outer sleeve to the inner sleeve to secure the stent graft to the shunt cannulae.

15. The device of claim 1, wherein the stentgraft is encapsulated in a polymer.

16. The device of claim 1, wherein the stentgraft and shunt are provided as separate components, and wherein the stentgraft and shunt are further adapted to be connected to create a single device.

17. A device, comprising:
a proximal flexible stentgraft having a distal tapered portion, the flexible stentgraft being encapsulated in a polymer; and
a distal shunt that includes a manifold and a one or a plurality of cannula connected to the manifold,
wherein the flexible stentgraft provides hemorrhage control while simultaneously providing blood inflow to the distal shunt and plurality of cannula.

18. The device of claim 17, further comprising a sheath that surrounds the flexible stentgraft and distal shunt, wherein the sheath peels-away during deployment of the device within a vessel.

19. The device of claim 17, further comprising:
a nosecone; and
a guidewire;
wherein the integrated guidewire tip is used to advance the device into a vessel.

20. The device of claim 19, further comprising a split introducer needle, wherein the guidewire is placed through the split introducer needle, and wherein pulling the guidewire splits the split introducer needle in half while the device is advanced into the vessel.

21. The device of claim 17, wherein the plurality of cannula provide blood flow to organs and predetermined target areas in a patient's body.
